(19) United States
(12) Patent Application Publication
(10) Pub. No.: US 2004/0220416 A1
(43) Pub. Date: Nov. 4, 2004

(54) Process for the singlet oxygen oxidation of organic substrates

(76) Inventors: Martin Emsenhuber, Linz (AT); Gerard Kwant, Sittard (NL); Koenraad Van Straaten, Echt-Susteren (NL); Madelon Janssen, Hasselt (BE); Paul Alsters, Maastricht (NL); Hendrik Hoving, Maastricht (NL)

Correspondence Address: WENDEROTH, LIND & PONACK, L.L.P., 2033 K STREET N. W., SUITE 800, WASHINGTON, DC, 20006-1021, US

(21) Appl. No.: 10/668,259

(22) Filed: Sep. 24, 2003

(30) Foreign Application Priority Data
Sep. 26, 2002 (AT) A 1443/2002

(51) Int. Cl. C07D 301/12

(52) U.S. Cl. 549/531

(57) ABSTRACT

Improved process for the oxidation of organic substrates by means of ¹O₂, in which 3-90% strength H₂O₂ is added to organic substrates which are soluble in water or in organic solvents miscible with water and which react with ¹O₂, in a water-miscible organic solvent, in water or in a mixture of water and water-miscible organic solvent in the presence of a heterogeneous or homogeneous catalyst, whereupon, following the catalytic decomposition of H₂O₂ to give water and ¹O₂, the oxidation of the substrates to give the corresponding oxidation products takes place, where, during the reaction, water is selectively removed from the reaction mixture by means of membranes.

The invention relates to an improved process for the singlet oxygen oxidation of organic substrates in which water is selectively removed from the reaction mixture during the reaction by means of membranes.

The only single oxygen oxidation (¹O₂ oxidation) which is currently carried out industrially is the photochemical ¹O₂ oxidation in which the ¹O₂ is generated by a photochemical route. The disadvantage of this process results from the high cost of the photochemical equipment required, and by a limited service life. The required lamps degenerate relatively rapidly during the oxidation as a result of soiling of the glass surface. In addition, this process is not suitable for coloured substrates. The process is actually suitable only for fine chemicals which are prepared on a relatively small scale. (La Chimica e l'Industria, 1982, Vol. 64, page 156).

For this reason, attempts have been made to find other process variants for the ¹O₂ oxidation in which ¹O₂ is generated chemically instead of photochemically.

J. Am. Chem. Soc., 1968, 90, 975 describes, for example, the classical “dark” ¹O₂ oxidation in which ¹O₂ is generated not photochemically, but chemically. In this process, hydrophobic substrates are oxidized by means of a hypochlorite/H₂O₂ system in a solvent mixture of water and organic solvent. Due to the secondary reactions between hypochlorite and substrate or solvent, the potential use of this process is somewhat limited. In addition, this process is not suitable for the industrial scale since addition of the hypochlorite onto H₂O₂ results in the organic medium, and a large excess of H₂O₂ is required to suppress the secondary reaction of substrate with hypochlorite. An additional disadvantage arises as the result of the formation of stoichiometric amounts of salt.

A variant of the dark ¹O₂ oxidation which is not based on hypochlorite and thus should partly avoid the above disadvantages, is known, for example, from J. Org. Chem., 1989, 54, 726 or J. Mol. Cat., 1997, 117, 439, according to which some water-soluble organic substrates are oxidized with ¹O₂ and a molybdate catalyst in water as solvent.

The disadvantage of the dark ¹O₂ oxidation known hitherto which is carried out in water or a mixture of water and organic solvent is, however, as described, for example, in J. Am. Chem. Soc. 1997, 119, p. 5286, that the majority of the ¹O₂ is lost as a result of quenching by the water molecules, meaning that a large excess of ¹O₂ is required in order to achieve acceptable conversions.

Since H₂O₂ is usually obtainable as an up to 30 or 35% strength aqueous solution and, additionally, water also arises during the formation of ¹O₂, reactions in which the singlet oxygen used as oxidizing agent is obtained in situ from ¹O₂ additionally suffer from additional dilution of the reaction medium with water. This also leads to reaction volumes being lost in the case of batch reactions, as a result of which the space-time yield is likewise reduced.

A further disadvantage of dark ¹O₂ oxidation in water or in water/solvent mixtures consists in the fact that, in the case of some organic substrates, in particular in the case of those with a relatively high molecular weight, demixing or phase separation arises when the water content of the reaction medium is high, which has a very negative effect on the yield.

Accordingly, it was an object of the present invention to find an improved method of the dark ¹O₂ oxidation which can be carried out simply, cost-effectively and in an environmentally friendly manner on the industrial scale, with the disadvantages which arise as a result of the presence of water being overcome.

Unexpectedly, it has now been found that the dark ¹O₂ oxidation can be carried out in an extremely efficient manner with a high yield in water or in water/solvent mixtures if water is selectively removed from the reaction mixture during the reaction by means of membranes.

Accordingly, the present invention provides an improved process for the oxidation of organic substrates by means of 102, which is characterized in that 3-90% strength H₂O₂ is added to organic substrates which are soluble in water or in organic solvents miscible with water and which react with ¹O₂, in a water-miscible organic solvent, in water or in a mixture of water and water-miscible organic solvent in the presence of a heterogeneous or homogeneous catalyst, whereupon, following the catalytic decomposition of H₂O₂ to give water and ¹O₂, the oxidation of the substrates to give the corresponding oxidation product takes place, where, during the reaction, water is selectively removed from the reaction mixture by means of membranes.

The process according to the invention is suitable for the oxidation of organic substrates which are water-soluble or soluble in water-miscible organic solvent and which react with ¹O₂.

Suitable substrates are described, for example, in WO 00/64842 and WO 00/61524. Excluded are those substrates which are highly hydrophobic such as, for example, rubrene, and those which are insoluble in water or in a water-miscible organic solvent.

Accordingly, the following compounds may be used as substrates: olefins which contain one or more, i.e. up to 10, preferably up to 6, particularly preferably up to 4, C═C double bonds; electron-rich aromatics, such as C₆-C₃₀-, preferably up to C₂₀-, particularly preferably up to C₁₅-aromatics, such as, for example, phenols, polyalkylbenzenes, polyalkoxybenzenes, etc.; polycyclic aromatics with 2 to 8, preferably up to 4, particularly preferably up to 3, aromatic rings; sulfides, such as, for example, alkyl sulfides, alkenyl sulfides, aryl sulfides which are either mono- or disubstituted on the sulfur atom, and heterocycles with one or more O, N or S atoms in the ring, such as, for example, C₄-C₃₀-, preferably up to C₂₀-, particularly preferably up to C₁₅-heterocycles, such as, for example, furans, pyrroles, thiophenes, thiazoles, pyridines, isobenzofurans, quinolines etc.

The substrates can here have one or more substituents, such as halogen, (F, Cl, Br, I), cyanide, carbonyl groups, hydroxyl groups, C₁-C₂₀, preferably up to C₁₀, particularly preferably up to C₆, alkoxy groups, C₁-C₂₀-, preferably up to C₁₀-, particularly preferably up to C₆-alkyl groups, C₆-C₃₀, preferably up to C₂₀, particularly preferably up to C₁₀, aryl groups, C₂-C₂₀, preferably up to C₁₀, particularly preferably up to C₆, alkenyl groups, C₂-C₂₀, preferably up to C₁₀, particularly preferably up to C₆, alkynyl groups, carboxylic acid groups, ester groups, amide groups, amino groups, nitro groups, silyl groups, silyloxy groups, sulfone groups, sulfoxide groups.

In addition, the substrates can be substituted by one or more NR¹R² radicals in which R¹ or R² may be identical or different and are H; C₁-C₂₀, preferably up to C₁₀, particullarly preferably up to C₆, alkyl; formyl; C₂-C₂₀, preferably up to C₁₀, particularly preferably up to C₆, acyl; C₇-C₃₀, preferably up to C₂₀, particularly preferably up to C₁₀, benzoyl, where R¹ and R² can also together form a ring, such as, for example, in a phthalimido group.

Examples of particularly suitable substrates are: 1,3-butadiene; 2,3-dimethylbutene; Δ^{9,10}-octalin, 2,3-dimethyl-1,3-butadiene; 2,4-hexadiene; 1,3-cyclohexadienes, 4-methyl-3-penten-2-ol, 3,4-dimethyl-3-penten-2-ol, 3-(4-methyl-1-naphthyl)propionic acid, 3,3′-(naphthalene-1,4-diyl)dipropionic acid, 1-trimethylsilylcyclohexene; (E)-2-methylcrotonic acid, 2,3-dimethyl-2-butenyl para-tolyl sulfone; 2,3-dimethyl-2-butenyl para-tolyl sulfoxide; N-cyclohexenylmorpholine; 2-methyl-2-norbornene; terpinolene; α-pinene; β-pinene; β-citronellol; ocimene; citronellol; geraniol; farnesol; terpinene; limonene; trans-2,3-dimethylacrylic acid, α-terpinene; isoprene; cyclopentadiene; 1,4-diphenylbutadiene; 2-ethoxybutadiene; 1,1′-dicyclohexenyl; cholesterol; ergosterol acetate; 5-chloro-1,3-cyclohexadiene; 3-methyl-2-buten-1-ol; 2-phthalimido-4-methyl-3-penten-2-ol, phenol, 1,2,4-trimethoxybenzene, 2,3,6-trimethylphenol, 2,4,6-trimethylphenol, 1,4-dimethylnaphthalene, furan, furfuryl alcohol, furfural, 2,5-dimethylfuran, isobenzofuran, dibenzyl sulfide, (2-methyl-5-tert-butyl)phenyl sulfide, diphenyl sulfide, etc.

The substrates can also be used in the form of a salt, for example in the form of the Na or K salt or in the form of the tetra-C₁-C₆-alkylammonium salt.

The corresponding oxidation product is obtained from the substrates by the oxidation according to the invention. Alkenes, (polycyclic) aromatics or heteroaromatics give, in particular, hydroperoxides or peroxides, iwhich can further react under the reaction conditions to give alcohols, epoxides, acetals or carbonyl compounds, such as ketones, aldehydes, carboxylic acids or esters, if the hydroperoxide or the peroxide is not stable.

The oxidation according to the invention takes place in a water-miscible organic solvent or in water or in a mixture of water and a water-miscible organic solvent. Suitable water-miscible solvents are C₁-C₈ alcohols, such as methanol, ethanol, propanol, isopropanol, butanol, isobutanol; ethylene glycol, propylene glycol, formamide, N-methylformamide, dimethylformamide (DMF), sulfolane, dioxane, THF and 1,2-dimethoxyethane. Preference is given to using methanol, ethanol, propanol, isopropanol, dioxane, DMF and THF, particular preference to using methanol, ethanol or THF as water-miscible solvent.

A metal which is suitable for ¹O₂ oxidations and is described, for example, in WO 00/64842 and WO 00/61524, J. Am. Chem. Soc., 1985, 107, 5844, Chem. Eur. J. (2001) 7(12), p. 2547-2556 or in Membrane Lipid Oxid. Vol. II, 1991, 65, is added as heterogeneous or homogeneous inorganic catalyst to the solvent-substrate mixture.

Preference is given to using catalysts based on molybdenum, tungsten, scandium, vanadium, titanium, zirconium, praseodymium, neodymium, samarium, europium, terbium, dysprosium, holmium, erbium, ytterbium, lanthanum, cerium or gadolinium, and also the so-called pseudo-lanthanides scandium and yttrium and lutetium. Particularly preference is given to molybdenum and lanthanum catalysts.

In this connection, the metal can be in forms customary for ¹O₂ oxidations, for example as the oxide, oxo complex, nitrate, carboxylate, hydroxide, layered double hydroxide (LDH), carbonate, chloride, flluoride, sulfate, tetrafluoroborate, etc. A hydroxide, for example NaOH, KOH, etc., can optionally be added to homogeneous, soluble forms of the catalyst to give a heterogeneous, active catalyst.

The amount of catalyst used depends on the substrate used and is between 1 and 50 mol%, preferably between 5 and 25 mol%.

In some instances, it may be advantageous, for better activation of the catalyst, if customary basic or acidic additives are added to the reaction mixture.

3-90% strength, preferably 30-60% strength, H₂O₂ is then added. Preferably, H₂O₂ is added slowly or in portions to the reaction mixture of solvent, substrate and catalyst, during which the reaction mixture is stirred. It is also possible to firstly add only some of the H₂O₂, then a hydroxide, such as, for example, NaOH, KOH etc., and then the remaining amount of H₂O₂.

The consumption of H₂O₂ in the process according to the invention is dependent on the substrate used. For reactive substrates, 2 to 3 equivalents of H₂O₂ are preferably required, whereas less reactive substrates are preferably reacted with 3 to 10 equivalents of H₂O₂.

The reaction temperature is between 0 and 50° C., preferably between 15 and 35° C.

The pH of the reaction mixture depends on the chosen substrate and the chosen catalyst and can be between 0 and 14, preferably between 4 and 14. The pH of the reaction mixture can, if necessary, be adjusted as required using customary basic or acidic additives.

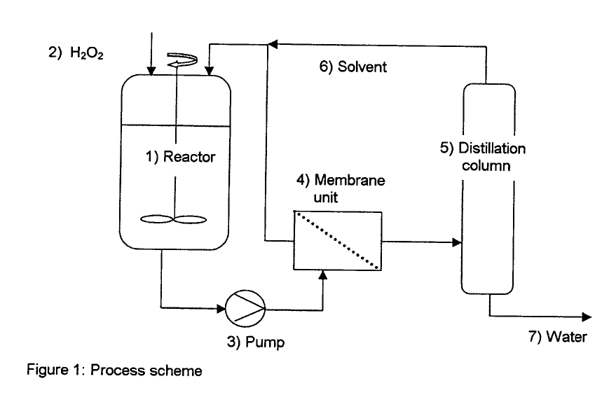

According to the invention, in the present process, water is selectively removed by means of membranes. In this connection, water which is optionally used as solvent and water which is introduced by the 2-90% strength H₂O₂ solution and which is formed during the catalysed disproportionation of H₂O₂, and optionally present water-miscible organic solvent is removed from the reaction mixture simultaneously by a membrane unit, whereupon distillative separation of the water from the organic solvent then optionally takes place. The organic solvent is then reintroduced into the reactor. The process scheme is shown in FIG. 1 and shows a reactor (1) in which the solvent or solvent mixture, the substrate and the catalyst are initially introduced and into which H₂O₂ is then introduced via line (2). Via a pump (3), the reaction mixture passes into the membrane unit (4). In the membrane unit (4), water (permeate) is then separated off through a suitable membrane, the catalyst (in the case of a homogeneous catalyst), the still unreacted substrate and product already formed being retained (retentate) and immediately reintroduced into the reactor.

Depending on the membrane chosen, the optionally used water-miscible organic solvent is either likewise retained, as is the case, for example, for MeOH for the use of inorganic membranes, or else is separated off with the water.

For the case that the organic solvent is separated off with the water from the reaction mixture, distillative separation (distillation column (5)) of water and water-miscible solvent then takes place, whereupon the solvent is returned to the reactor (line (6)) and the separated-off water is discarded.

In the process according to the invention, instead of one membrane unit, it is also possible to use two or more membrane units. If two membrane units are used, the permeate of the first unit is passed to the second membrane unit, and the retentate of the first membrane unit is returned, as described above, to the reactor. The retentate of the second unit is then likewise returned to the reactor, while the permeate of the second unit is worked up as described above (removal of the solvent by distillation) or, if the permeate comprises only water, is discarded.

For the process according to the invention, both organic and also inorganic membranes are suitable.

Suitable organic membranes are membranes for reverse osmosis (R.O.) which consist, for example, of polyvinyl alcohol, polyamide or polysulfone or cellulose acetate, cellulose diacetate, cellulose triacetate, cellulose diacetate/cellulose triacetate (CDA/CTA), cellulose nitrate, polypropylene, polyimide, sulfonated polysulfones, polyethersulfones, polyacrylonitrile, polyimide/polyetherimide, polyvinylidene fluoride, aramid or polypiperazine or mixtures thereof.

Preference is given to those membranes which retain molecules with a molecular weight between 50 and 200 Da (g/mol), particularly preferably between 50 and 100 Da.

Further suitable organic membranes are membranes for nanofiltration whose active separation layer comprises, for example, polyvinyl alcohol, polypropylene, polysulfone, polyether-sulfone, polyacrylonitrile, polyimide/polyether-imide, polyvinylidene fluoride, polyamide, polypiperazine, cellulose acetate, cellulose nitrate, aramide, cellulose diacetate, cellulose triacetate, etc. or mixtures thereof.

Preference is given to those nanofiltration membranes which retain molecules with a molecular weight between 50 and 400 Da (g/mol), particularly preferably between 50 and 200 Da.

As organic membranes, preference is given to using R.O. membranes, which are in fact suitable for all suspended and dissolved materials since these are all retained.

Commercially available R.O. membranes are, for example, Toray SU810 (Toray, based on polyamide), PCI ACF99 (PCI, based on polyamide), DESAL SC (Osmonics-Desalination Systems, based on cellulose acetate), Pall Rochem 05757, SW 30 HR (Film Tec-DOW, based on polyamide), Trisep X-20 (Trisep, based on ACM/cellulose acetate), NTR 759 (Nitto Denko; based on polyvinyl alcohol/polyamide), NTR 729 (Nitto Denko; based on polyvinyl alcohol); Pervap 1510 (Deutsche Carbone AG), CMC-CE01 (Celfa, based on polyvinyl alcohol) etc.

Commercially available N.F. membranes are, for example, Desal 5K (Osmonics-Desalination Systems; based on polyamide), Koch MPF60 (Koch-Membrane Products), NF200 (Filmtec DOW; based on polypiperazinamide), NF CA30 (Nadir; based on cellulose acetate) etc.

In addition, suitable organic membranes are also organic pervaporation membranes whose active separation layer comprises polydimethylsiloxanes, poly(1-trimethylsilyl-1-propyne), polyurethanes, poly-butadiene, polyether block polyamides, silicone polycarbonates, styrene-butadiene rubber, nitrile-butadiene rubber, ethene-propene terpolymer, polyvinyl alcohol, polyamide, polysulfone, cellulose acetate, aramid, cellulose diacetate, cellulose triacetate, polypiperazine etc. or mixtures thereof.

Organic pervaporation membranes are characterized in that they effectively separate water from molecules which are larger than 4.5 Å. They are preferably used for separating off molecules greater than 5 Å in size.

The inorganic or ceramic membranes used are preferably membranes for the pervaporation technique. These are membranes which consist, for example, of an active layer based on aluminium, titanium dioxide, silica, boron oxide, magnesium, zirconium, clay etc. or mixtures thereof, on a suitable carrier, such as, for example, a gamma-Al₂O₃ carrier.

Commercially available pervaporation membranes are, for example, Sulzer Pervap SMS (Sulzer, silica membrane system).

These inorganic pervaporation membranes are also characterized by the fact that they effectively separate water from molecules which are larger than 4.5 Å. They are preferably used for separating off molecules greater than 5 Å in size.

Also suitable are ceramic nanofiltration membranes based on aluminium, boron oxide, magnesium, zirconium, TiO₂, SiO₂, clay etc. or mixtures thereof, for example from HITA (Hermsdorfer Institut fur Technische Keramik [Hermsdorf Institute of Technical Ceramics]), and TAMI (Tami Industries) etc., and zeolite membranes based on aluminium, boron oxide, magnesium, zirconium, TiO₂, SiO₂, clay etc. or mixtures thereof.

Preference is given to using ceramic membranes for the process according to the invention.

The most important parameters for suitable membranes here are:

capacity: permeate flow, kg/m².h.bar

selectivity: retention or separation factor,

mechanical stability: abrasion, temperature

chemical stability: towards solvents and oxidising agents

Suitable membranes should have a retention factor for substrate and product of at least 85%, preferably of at least 95%, in order to keep substrate and product losses as low as possible.

If two membrane units are used, then membranes with a relatively low retention factor of at least 80%, preferably of at least 90%, can be used.

The choice of a suitable membrane is made depending on the chosen reaction medium, substrate, product and catalyst.

Preferably, suitable membranes are ascertained in preliminary experiments in which the stability in the reaction medium, the retention factor with regard to substrate and product, the capacity (permeate flow) etc. are investigated.

The process according to the invention avoids the reaction mixture becoming increasingly diluted by the water introduced with the hydrogen peroxide (through the use of a 3-90% strength solution and by disproportionation to water and ¹O₂). As a result, losses in yield in the case of the batch procedure and negative influences on the solubility (demixing etc.) and the efficiency of the ¹O₂ are prevented. In addition, negative influences of water on the stereoselectivity of the reaction are prevented.

As a result of the water removal according to the invention, it is accordingly possible to increase the yields in the case of the batch procedure by up to more than 200%, and to increase the stereoselectivity of the reaction.

Comparative Experiment: Without Water Removal

A mixture of methanol, 2-methylcrotonic acid and Na₂MoO₄ catalyst was initially introduced into a reactor. A 30% strength by weight H₂O₂ solution was then added. The batch volume increased as a result of the water which formed during the catalysed disproportionation of H₂O₂, and by the volume of water in the 30% strength by weight H₂O₂ solution. The total volume at the end of the batch experiment determines the maximum initial charging of the reactor or the batch yield at the end of the reaction.

Table 1 gives the reaction parameters

| | % water at end | | |
|---|---|---|---|
| H₂O₂/substrate | of batch | [substrate] | Batch yield |
| mol/mol | % by wt. | mol/l | kg |
| 2 | 5.0 | 0.22 | 196 |
| 2 | 30.0 | 1.93 | 1333 |
| 2 | 18.9 | 1 | 795 |
| 2 | 49.5 | 5.1 | 2431 |
| 10 | 5.0 | 0.04 | 39 |
| 10 | 30.0 | 0.36 | 251 |
| 10 | 53.9 | 1 | 481 |
| 10 | 84.9 | 5 | 810 |

### EXAMPLE 1

### Water Removal Using a Membrane Unit

A mixture of methanol, substrate and Na₂MoO₄ catalyst was initially introduced into a reactor. A 30% strength by weight H₂O₂ solution was then added. The batch volume increased by the water which formed during the catalysed disproportionation of H₂O₂, and by the volume of water in the 30% strength by weight H₂O₂ solution.

After the previously determined total content of water had been reached, the membrane unit (Celfa) was set in operation in order to remove further water which was added and which formed. Likewise removed methanol was completely recovered by distillative separation and returned to the reactor.

TABLE 2
Batch yield for a readily oxidizable
substrate, such as, for example, β-citronellol with an
H₂O₂/substrate molar ratio = 2;

| | Water | Batch | % increase | |
|---|---|---|---|---|
| | content | yield | compared with | |
| Retention | % by wt. | kg | Comp. Ex. | Yield % |
| 90 | 5 | 609 | 211 | 64 |
| 95 | 5 | 759 | 287 | 80 |
| 99 | 5 | 908 | 363 | 96 |
| 100 | 5 | 950 | 385 | 100 |

TABLE 3
Batch yield for a substrate which is difficult
to oxidize, such as, for example, tiglic acid with an
H₂O₂/substrate molar ratio = 10;

| | Water | Batch | % increase | |
|---|---|---|---|---|
| | content | yield | compared with | |
| Retention | % by wt. | kg | Comp. Ex. | Yield % |
| 90 | 5 | 91 | 133 | 10 |
| 95 | 5 | 291 | 646 | 31 |
| 99 | 5 | 748 | 1818 | 78 |
| 100 | 5 | 950 | 2336 | 100 |
| 90 | 30 | 677 | 170 | 71 |
| 95 | 30 | 801 | 219 | 84 |
| 99 | 30 | 917.8 | 266 | 96.6 |
| 100 | 30 | 950 | 278 | 100 |

### EXAMPLE 2

### Water removal with two membrane units

The experiment was carried out analogously to Example 1, although two membrane units were used to remove the water.

TABLE 4
Batch yield for a readily oxidizable substrate
with an H₂O₂/substrate molar ratio = 2;

| | Water | Batch | % increase | |
|---|---|---|---|---|
| | content | yield | compared with | |
| Retention | % by wt. | kg | Comp. Ex. | Yield % |
| 90 | 5 | 898 | 358 | 95 |
| 95 | 5 | 937 | 378 | 98.6 |
| 99 | 5 | 949.5 | 384 | 99.94 |
| 100 | 5 | 950 | 385 | 100 |

TABLE 5
Batch yield for a substrate which is difficult
to oxidize with an H₂O₂ substrate molar ratio - 10;

| | Water | Batch | % increase | |
|---|---|---|---|---|
| | content | yield | compared with | |
| Retention | % by wt. | kg | Comp. Ex. | Yield % |
| 90 | 5 | 707 | 1713 | 75 |
| 95 | 5 | 881 | 2159 | 93 |
| 99 | 5 | 947 | 2328 | 99.69 |
| 100 | 5 | 950 | 2336 | 100 |
| 90 | 30 | 912 | 262 | 96 |
| 95 | 30 | 940 | 275 | 98 |
| 99 | 30 | 949.6 | 278 | 99.96 |
| 100 | 30 | 950 | 278 | 100 |

### EXAMPLE 3

It was investigated whether organic R.O. membranes are suitable for the removal of a water/methanol mixture coupled with simultaneously high retention of substrate and product:

In an experimental plant according to FIG. 1, a test solution of 7% by weight of Na tiglate, 5% by weight of water and 88% by weight of methanol was pumped at 25° C. and a pressure difference of 30 bar at the membrane from the reactor using a pump through the membrane unit (R.O. membrane, type NTR 759), and the retention, and also the flow, were determined.

Beforehand, the membrane was stored for 5 days at room temperature and atmospheric pressure for stabilisation in 5% by weight of water/methanol.

Result: 96% retention of Na tiglate, 2 kg/m²/h flow

### EXAMPLE 4

It was investigated whether organic R.O. membranes are stable under singlet oxygen oxidation conditions:

In an experimental plant according to FIG. 1, a test solution of 7% by weight of Na tiglate, 5% by weight of water and 88% by weight of methanol was pumped at 25° C. and a pressure difference of 30 bar at the membrane by means of a pump from the reactor through the membrane unit (R.O. membrane, type NTR 759), and the retention, and also the flow were determined.

Beforehand, the membrane was exposed for 5 days to singlet oxygen oxidation conditions: 5% by weight of water/methanol, pH about 10, 0.02M Na₂MoO₄, H₂O₂ addition at an H₂O₂/catalyst molar ratio of 3, room temperature, atmospheric pressure; the solution was renewed 6 times per day.

Result: 85% retention of Na tiglate, 13-16 kg/m²/h flow

### EXAMPLE 5

It was investigated whether a ceramic pervaporation membrane is suitable for the removal of a water/methanol mixture with simultaneously high retention of substrate and product:

In an experimental plant according to FIG. 1, a test solution of 7% by weight of Na tiglate, 5% by weight of water and 88% by weight of methanol was pumped at 25° C. and a pressure difference of 7 bar at the membrane by means of a pump from the reactor through the membrane unit (Sulzer Pervap SMS), and the retention, and also the flow were determined.

Result: 100% retention of Na tiglate, 0.1 kg/m²/h flow; water/methanol selectivity =6

1. Improved process for the oxidation of organic substrates by means of ¹O₂, characterized in that 3-90% strength H₂O₂ is added to organic substrates which are soluble in water or in organic solvents miscible with water and which react with ¹O₂, in a water-miscible organic solvent, in water or in a mixture of water and water-miscible organic solvent in the presence of a heterogeneous or homogeneous catalyst, whereupon, following the catalytic decomposition of H₂O₂ to give water and ¹O₂, the oxidation of the substrates to give the corresponding oxidation products takes place, where, during the reaction, water is selectively removed from the reaction mixture by means of membranes.

2. Improved process according to claim 1, characterized in that the substrates used which react with ¹O₂ are olefins which contain 1 to 10 C═C double bonds; C₆-C₃₀ aromatics; polycyclic aromatics having 2 to 8 aromatic rings; alkyl sulfides, alkenyl sulfides, aryl sulfides which are either mono- or disubstituted on the sulfur atom, and C₄-C₃₀ heterocycles with one or more 0, N or S atoms in the ring, which may be unsubstituted or may be mono- or polysubstituted by halogens, cyanide, carbonyl groups, hydroxyl groups, C₁-C₂₀ alkoxy groups, C₁-C₂₀ alkyl groups, C₆-C₃₀ aryl groups, C₂-C₂₀ alkenyl groups, C₂-C₃₀ alkynyl groups, carboxylic acid groups, ester groups, amide groups, amino groups, nitro groups, silyl groups, silyloxy groups, sulfone groups, sulfoxide groups or by one or more NR¹R² radicals in which R¹ or R² may be identical or different and are H; C₁-C₂₀ alkyl; formyl, C₂-C₂₀ acyl; C₇-C₃₀ benzoyl, where R¹ and R² may also together form a ring.

3. Improved process according to claim 1, characterized in that the organic water-miscible solvent used is C₁-C₈-alcohol, ethylene glycol, propylene glycol, formamide, N-methylformamide, dimethylformamide, sulfolane, dioxane, THF or 1,2-dimethoxyethane.

4. Improved process according to claim 1, characterized in that catalysts based on molybdenum, tungsten, scandium, vanadium, titanium, zirconium, praseodymium, neodymium, samarium, europium, terbium, dysprosium, holmium, erbium, ytterbium or lutetium in the form of oxides, oxo complexes, nitrates, carboxylates, hydroxides, layered double hydroxides, carbonates, chlorides, fluorides, sulfates or tetrafluoroborates are used.

5. Improved process according to claim 1, characterized in that 2 to 10 equivalents of H₂O₂ are used depending on the substrate used.

6. Improved process according to claim 1, characterized in that the reaction temperature is between 0 and 50° C.

7. Improved process according to claim 1, characterized in that organic or inorganic membranes are used for the selective removal of water.

8. Improved process according to claim 7, characterized in that the organic membranes used for the reverse asmosis are membranes based on polyvinyl alcohol, polyamide or polysulfone or cellulose acetate, cellulose diacetate, cellulose triacetate, cellulose diacetate/cellulose triacetate, cellulose nitrate, polypropylene, polyimide, sulfonated polysulfones, polyether-sulfones, polyacrylonitrile, polyimide/polyether-imide, polyvinylidene fluoride, aramid or polypiperazine or mixtures thereof or nanofiltration membranes based on polyvinyl alcohol, polypropylene, polysulfone, polyether-sulfone, polyacrylonitrile, polyimide/polyether-imide, polyvinylidene fluoride, polyamide, polypiperazine, cellulose acetate, cellulose nitrate, aramid, cellulose diacetate, cellulose triacetate or mixtures thereof or organic pervaporation membranes based on polydimethylsiloxane, poly(1-trimethylsilyl-1-propyne), polyurethanes, polybutadiene, polyether block polyamides, silicone polycarbonates, styrene-butadiene rubber, nitrile-butadiene rubber, ethene-propene terpolymer, polyvinyl alcohol, polyamide, polysulfone, cellulose acetate, aramid, cellulose diacetate, cellulose triacetate, polypiperazine or mixtures thereof.

9. Improved process according to claim 7, characterized in that ceramic pervaporation, zeolite or nanofiltration membranes are used.

10. Improved process according to claim 1, characterized in that the membranes used have a retention factor for substrate and product of at least 85%.

11. Improved process according to claim 1, characterized in that, during the reaction, water which is optionally used as solvent and water which is introduced by the 3-90% strength H₂O₂ solution and which is formed during the catalyzed disproportionation of H₂O₂ is selectively removed from the reaction mixture by pumping the reaction mixture from a reactor (1) via a pump (3) into the membrane unit (4) in which then the water is separated off through a suitable membrane, the catalyst, in the case of a homogeneous catalyst, the still unreacted substrate and product already formed being retained and immediately reintroduced into the reactor (1) and, depending on the membrane chosen, the optionally used water-miscible organic solvent either likewise being retained, or else separated off with the water, whereupon distillative removal of the water from the organic solvent then takes place in the distillation column (5), the organic solvent is then reintroduced into the reactor (1) via the line (6), and the water which has been separated off is discarded.

12. Improved process according to claim 1, characterized in that instead of one membrane unit (4), two membrane units are used, where the permeate of the first unit is passed to the second membrane unit, and the retentate of the first membrane unit is returned to the reactor, and then the retentate of the second unit is likewise reintroduced into the reactor, while the organic water-miscible solvent is optionally distilled off from the permeate of the second unit or, if the permeate comprises only water, it is discarded.
